(19) United States
(12) Patent Application Publication
(10) Pub. No.: US 2026/0194461 A1
(43) Pub. Date: Jul. 9, 2026

(54) Refractometric System for Conducting Urinalysis in a Toilet

(71) Applicant: Vivosens Inc., San Francisco, CA (US)

(72) Inventors: Miray Tayfun, Istanbul (TR); Balim Bangisu Caf, Istanbul (TR); Haluk Celik, Istanbul (TR); Gizem Cebi, Istanbul (TR)

(21) Appl. No.: 19/010,146

(22) Filed: Jan. 5, 2025

(51) Int. Cl. G01N 21/41; E03D 9/00; G01N 33/493

(52) U.S. Cl. CPC G01N 21/4133; E03D 9/00; G01N 33/493; G01N 2021/414

(57) ABSTRACT

A toilet-mountable refractometric system for non-invasive urinalysis is disclosed. The system is designed to measure the specific gravity (SG), pH, ketone bodies, osmolality, glucose levels, sodium, electrolyte concentration, urine concentration, and dilution of urine to assess hydration status and kidney function. The device, which can be mounted in a toilet, operates by detecting changes in the refractive index of urine. A light source, optical sensor, and prism are configured within the system to facilitate measurement without direct handling of urine samples. Data is transmitted to a mobile application via Bluetooth and/or Wi-Fi, allowing users to receive real-time results post-analysis.

### TECHNICAL FIELD

This invention relates to the field of urinalysis systems, specifically to a refractometric device designed for in-toilet installation, capable of measuring a range of urine parameters. In addition to specific gravity, the device can assess hydration levels and kidney function by analyzing other urine components such as pH, protein, salinity, magnesium, calcium, vitamin C, ketones, and oxidative stress markers.

### BACKGROUND OF THE INVENTION

Urine specific gravity (USG) is a critical biomarker for hydration status and kidney function. Traditional methods for assessing USG in clinical settings often require sample handling in a laboratory. This refractometric system provides a non-invasive, quick, and efficient solution by allowing users to assess their hydration status in real-time. Existing urinalysis systems require manual sample collection or chemical reagents. However, this system utilizes refractometry for a more automated and user-friendly approach, promoting consistent health monitoring at home.

### BRIEF SUMMARY OF THE INVENTION

This invention provides a system and method for measuring the specific gravity of urine using refractometry in a toilet-mounted device. The system consists of the following components:

Light Source: A stable light source within the device that illuminates the urine sample to facilitate refractometric analysis.

Optical Sensor and Prism: The light passing through the urine sample is refracted at an angle dependent on the concentration of dissolved substances. The refractive index is then captured by the optical sensor, and the SG is computed.

Microcontroller/Processor: A microcontroller processes the data obtained from the sensor and calculates the specific gravity.

Temperature Sensor: This sensor adjusts the SG reading for variations in urine temperature to ensure accuracy.

Bluetooth and/or Wi-Fi Module: Enables wireless data transmission to a paired mobile application, which displays the SG reading to the user.

Passive Sample Collection and Removal Mechanism: The system employs a passive, non-mechanical and/or non-electronic sample collection and removal mechanism integrated into the device's plastic shell. This mechanism is designed to simplify the collection of urine samples while ensuring efficient disposal, all without the need for mechanical and/or electronic and/or motorized components. The mechanism does not require energy consumption.

The device includes strategically placed, small collection channels molded directly into the plastic shell. These channels are positioned to capture and retain a sample of urine as it passes through the device. The design and placement of the channels are optimized to allow gravity to guide urine into them during use, eliminating the need for active, powered movement.

Each collection channel is connected to a network of flow channels designed to direct excess urine away from the sensitive optical components of the refractometer. The channels are angled to allow gravity to naturally pull urine out of the wells and into a safe exit path. This design prevents overflow and minimizes potential contamination of internal sensors.

After the measurement is complete, the passive mechanism leverages gravity to drain the urine from the collection wells through a built-in drainage pathway. The exit pathways are constructed to automatically flush residual urine out of the device, making the collection process clean, reliable, and maintenance-free.

By using passive mechanisms rather than motorized parts, this system minimizes the risk of sample cross-contamination. The one-time collection and drainage design ensures each sample is isolated during the analysis process. This passive approach also reduces wear and tear on the device, enhancing durability.

The passive sample collection and removal mechanism in this refractometric urinalysis device provides a low-maintenance, efficient solution to urine sampling. It ensures accuracy by isolating samples during each measurement and promotes ease of use by automating the drainage process via gravity alone. This design reduces power consumption and mechanical complexity, thereby enhancing device longevity and reliability for daily hydration and kidney health monitoring.

### BRIEF DESCRIPTION OF THE DRAWINGS

To aid in understanding the disclosure and illustrate how it may be implemented in practice, embodiments will now be described as non-limiting examples, with reference to the accompanying drawings:

FIG. 1 is a diagram of the refractometric urinalysis system setup in a toilet using a hanger;

FIG. 2 is an exploded view of the smart toilet device components; and

FIG. 3 is an assembled view of the smart toilet device showing its key components.

The drawings described herein are provided for illustrative purposes and are not intended to limit the scope of the present disclosure in any way.

### DETAILED DESCRIPTION

In the figures, the following reference numerals denote components of the invention:
1. Front Part with Urine Holder—The outer casing, designed to collect urine for analysis.
2. PCB—The electronic control unit, housing the main processing and sensor components.
3. Lens—An optical component that facilitates precise image capture for urine analysis.
4. Back Cover with Battery Compartment—The rear part of the device, providing space for battery installation.
5. Battery Cover—A protective cover that secures the battery within its compartment.
6. Hanger for Mounting on a Toilet Closet - A structural element enabling the device to be affixed securely on a toilet closet for non-contact measurements.
7. LED Light—An indicator light to signal device status during operation and testing.
8. Urine Holder Funnel for Test—A funnel-like structure designed to guide urine into the detection area for analysis.

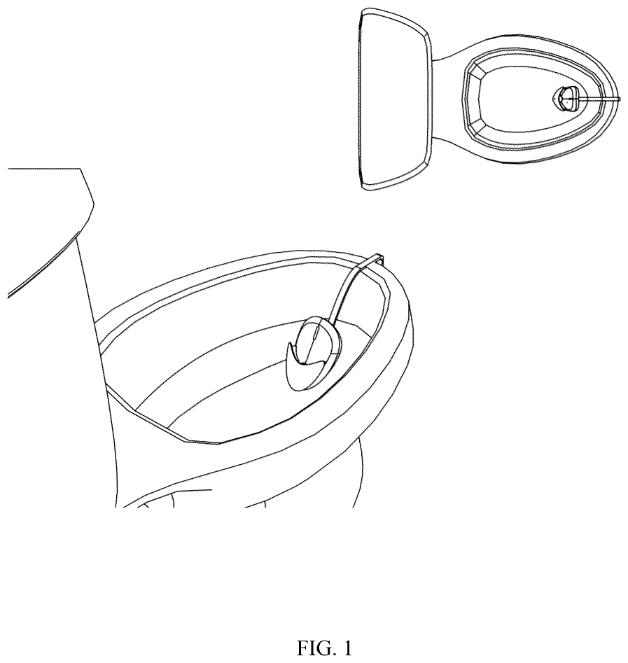

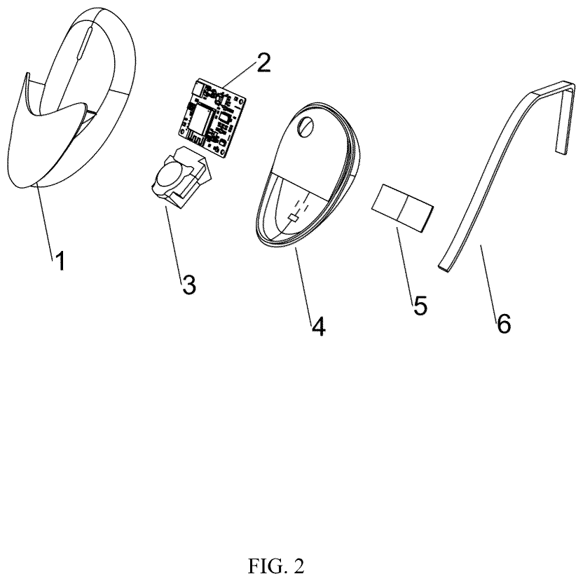

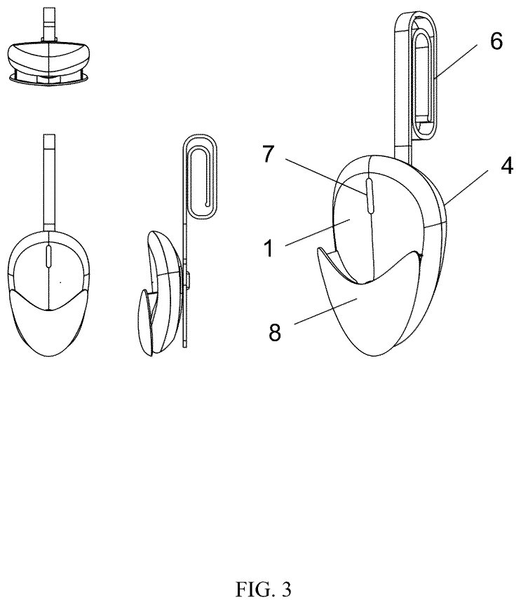

Referring to FIG. 1, a general overview of the refractometric urinalysis system is shown in a toilet-mounted configuration using a hanger 6. FIG. 2 illustrates an exploded view of the smart toilet device components, and FIG. 3 shows an assembled perspective highlighting the key structural elements.

As depicted, the device comprises a Front Part with Urine Holder (sometimes referred to simply as the “front part”), indicated generally at reference numeral 1. This portion provides an outer casing designed to receive urine passively for analysis. In one embodiment, the front part further comprises a Urine Holder Funnel for Test 8, which directs urine toward the detection area in an optimal manner, ensuring sufficient sample volume for accurate refractometric measurement.

A Printed Circuit Board (PCB) 2 is housed within the device, functioning as the central control unit. This PCB typically hosts the main processing circuitry, including one or more microcontrollers for data acquisition and computation. In communication with the microcontroller is a temperature sensor (not separately numbered but contained on or near the PCB), which adjusts the specific gravity reading based on the measured urine temperature.

An Optical Sensor and Prism assembly (collectively incorporated into or adjacent to the PCB) detects the angle of refraction from an internally mounted Light Source. Although the light source is not specifically numbered in the figures, it may be collocated near or on the PCB for ease of alignment. The system also employs a Lens 3, arranged to focus or direct light onto the optical sensor to enable precise image capture or refractometric measurement.

Behind the front part, a Back Cover with Battery Compartment 4 encloses a dedicated space for power storage. A Battery Cover 5 is provided to secure the battery in place, protecting it from moisture and accidental dislodgement. This arrangement allows for quick battery replacement while shielding internal electronics from fluid ingress.

A Hanger for Mounting on a Toilet Closet 6 (or equivalent mounting structure) permits easy attachment and detachment of the entire device to a toilet, enabling stable positioning for non-contact measurements. The hanger can be designed to hook onto or clamp around the toilet rim or flush tank, depending on user preference and toilet geometry.

An LED Light 7 provides visual feedback for operational status, such as power-up, pairing with a mobile device, test initiation, and completion. The LED may blink or change color to convey different device states, aiding users in following prompts from a companion smartphone application.

The system's Passive Sample Collection and Removal Mechanism is molded directly into the device's plastic shell. It comprises small collection channels positioned in such a way that urine is captured by gravity flow during urination. No mechanical, electronic, or motorized components are involved in sample collection or drainage. Instead, each collection channel is connected to a network of flow channels, angling downward to direct excess fluid away from the optical components. Post-measurement, residual urine drains automatically via these channels, minimizing contamination and eliminating the need for powered cleaning systems.

By harnessing gravity for sample capture and removal, this passive mechanism significantly reduces device complexity and potential points of failure. It also helps isolate each sample and prevents cross-contamination between subsequent uses. The mechanism requires no additional energy consumption, making it suitable for frequent or daily monitoring while maintaining long battery life.

The refractometric urinalysis system operates as follows:

### 1. Mounting and Setup

Once the user unpacks the device, it is mounted onto a toilet using clips, suction cups, or the provided hanger 6. The device remains stable during normal use. The user then pairs the device with a mobile application (e.g., Vivoo App) via Bluetooth and/or Wi-Fi. This initial setup ensures that the user's smartphone can communicate wirelessly with the device.

### 2. Operation and Measurement

Before urination, the user opens the Vivoo App on a paired smartphone to activate or “wake” the device. The application may provide on-screen instructions guiding the user to position themselves properly so that urine flows naturally into the collection channels. During urination, a passive sample collection occurs through the molded channels in the Front Part with Urine Holder 1 and/or Urine Holder Funnel for Test 8. Once the sample is captured, the user initiates the urinalysis from the Vivoo App. A built-in light source illuminates the urine; as the light traverses the fluid, it refracts at an angle related to the solute concentration. The Optical Sensor and Prism assembly, coupled with the Lens 3, captures this refraction angle. The onboard microcontroller/processor on the PCB 2 interprets the data to calculate the urine's specific gravity. Finally, the result is sent to the smartphone application via Bluetooth or Wi-Fi.

### 3. Temperature Compensation

An integrated temperature sensor (housed near or on the PCB) measures the temperature of the collected urine sample. Variations in temperature can affect refractometer readings, so the microcontroller automatically applies compensation algorithms to ensure accuracy. The corrected specific gravity (SG) value reflects the true solute concentration of the urine.

### 4. Data Transmission

After computation, the device's wireless module transmits the SG reading to the paired Vivoo App. The transmitted data typically includes the SG value and may also integrate additional analysis parameters if the system assesses other biomarkers such as pH, protein content, salinity, magnesium, calcium, vitamin C, ketones, or oxidative stress markers. Over time, these readings help users monitor hydration and kidney health trends directly from the app.

### 5. Cleaning Mechanism

The same collection channels used for urine sampling also facilitate cleaning of the analysis well. After each test, the user simply flushes the toilet in the normal manner. Water from the toilet flush flows through the channels, rinsing residual urine from the device's interior surfaces. Excess fluid then exits through a small hole in the device shell, draining away safely to reduce any risk of buildup. Because no mechanical or motorized parts are required, this passive cleaning mechanism is reliable, maintenance-free, and power-efficient.

### How to Use

### 1. Download the Vivoo Mobile App:

Follow the app's instructions to locate the device name (e.g., “Vivoo Hydration Smart Toilet”) in the Bluetooth or Wi-Fi settings.
Select the device and pair it.
During pairing, the LED Light 7 will blink blue for approximately 3 seconds to indicate a successful connection.

### 2. Open the Mobile App and Press the “Start Test” Button:

Once “Start Test” is tapped, the LED Light 7 blinks green rapidly.
Follow the on-screen prompts to begin urination.
After urination, the user selects an option within the app to initiate the analysis process.

### 3. The Device Analyzes the Urine:

The system performs refractometric measurement(s) of the specific gravity, along with any additional parameters (e.g., pH or ketones) that the design enables.
Hydration levels are determined accordingly, and results are displayed in the mobile app.

### 4. Completing the Test:

When the measurement concludes, the user presses the “Finish Test” button within the Vivoo App.
   The LED Light 7 turns off, indicating that the process is complete.
   The app instructs the user to flush the toilet, thereby triggering the passive cleaning mechanism described above. The device is then ready for the next measurement.

Although specific embodiments have been described in connection with the figures, it will be understood by those skilled in the art that the disclosed invention is not limited to the particular forms shown. Modifications and alternatives may be made without departing from the scope of the present disclosure, which is defined by the appended claims.

I claim:

1. A toilet-mountable refractometric system for non-invasive urinalysis, comprising:
a light source configured to illuminate a urine sample;
an optical sensor and prism arranged to detect changes in the refractive index of the urine sample based on light refraction;
a microcontroller programmed to process data from the optical sensor and calculate the specific gravity (SG) of the urine sample;
a memory module to store previous measurement data;
a passive sample collection and removal mechanism, comprising integrated collection channels and flow channels to guide and drain urine by gravity alone;
a wireless communication module for data transmission to the Vivoo mobile application via Bluetooth and/or Wi-Fi;
an LED indicator light configured to change color to indicate system status, including green for Bluetooth connection, blue for standby mode, and red for low battery;
a rechargeable and/or replaceable lithium-ion battery, wherein the device operates in a water-resistant housing with an antimicrobial nano-coating, both internally and externally, to protect against water, humidity, and microbial contamination;
several urine collection channels to collect urine and self-cleaning using water from a toilet flush; and,
a housing mountable within a toilet using clips and/or suction cups.

2. The system of claim 1, further comprising a standby mode that allows the device to conserve power until the user connects via Bluetooth and/or Wi-Fi, at which point the device activates for urinalysis.

3. The system of claim 1, wherein the memory module stores data from multiple previous urinalysis sessions, enabling tracking of historical hydration status and kidney function via a mobile application.

4. The system of claim 1, wherein the antimicrobial nano-coating includes both interior and exterior surfaces, preventing microbial buildup and ensuring hygienic operation over extended periods.

5. The system of claim 1, wherein the LED indicator light provides real-time feedback to the user, displaying different colors corresponding to device status and alerts, including connection status, standby mode, and low battery.

6. The system of claim 1, wherein the water-resistant housing protects sensitive internal components from water and humidity exposure during operation in a toilet-mounted environment.

7. The system of claim 1, further comprising a temperature sensor operatively connected to the microcontroller, wherein the temperature sensor adjusts the SG reading according to the urine temperature to enhance measurement accuracy.

8. The system of claim 1, wherein the rechargeable lithium-ion battery provides sufficient power for repeated urinalysis operations, and the battery is replaceable to extend device longevity.

9. The system of claim 1, wherein the wireless communication module is further configured to synchronize stored historical data from the device memory with the mobile application upon connection.

10. The system of claim 1, wherein the specific gravity values obtained range from 1.005 to 1.060, corresponds to hydration levels from well-hydrated to seriously dehydrated.

11. The system of claim 1, wherein the Bluetooth module transmits specific gravity readings to the mobile application, which categorizes hydration levels as well-hydrated, minimal dehydration, significant dehydration, and serious dehydration.

12. The system of claim 1, wherein the passive sample collection and removal mechanism is designed to drain residual urine and prevent buildup of contaminants, while the antimicrobial nano-coating enhances protection against microbial contamination.

13. The system of claim 1, further comprising an in-toilet mounting assembly that stabilizes the device for accurate measurements without manual urine handling.

14. The system of claim 1, wherein each collection channel is linked to a network of flow channels angled to direct excess urine away from the optical sensor and prism, thereby preventing overflow and reducing contamination risk of internal components.

15. The system of claim 1, wherein the microcontroller is further configured to analyze urine components beyond specific gravity, including pH, protein levels, salinity, magnesium, calcium, vitamin C, ketones, and oxidative stress markers.

16. The system of claim 1, further comprising a cleaning mechanism utilizing water from the toilet flush, wherein the water flow passively cleans the collection channels and analysis well, with remaining water draining from an exit hole in the device shell.

17. The system of claim 1, wherein the device includes mounting hardware such as clips or suction cups for stable attachment within a toilet, enabling accurate and consistent sample collection during each use.

18. A method for conducting urinalysis using a toilet-mountable refractometric system, comprising:
mounting the device within a toilet,
waiting in standby mode until the user connection via Bluetooth and/or Wi-Fi,
using gravity to collect a urine sample in a passive collection channel and performing refractometric analysis,
calculating specific gravity (SG) and adjusting for urine temperature using a temperature sensor,
storing the SG data in a memory module, and
transmitting SG data and historical data from memory to a mobile application for tracking hydration and kidney health.

19. The method of claim 18, further comprising activating an LED indicator light to signal system status, including Bluetooth connection, standby, and low battery, and passively cleaning the collection channels by directing flush water through the device.
